(19) United States
(12) Patent Application Publication
(10) Pub. No.: US 2025/0107944 A1
(43) Pub. Date: Apr. 3, 2025

(54) SIMULTANEOUS RESISTANCE AND CAPACITANCE MEASUREMENT AND INFERENCING ALGORITHM ENABLING REAL TIME AND IMPROVED INCONTINENCE WET SENSING AND DIAPER SATURATION ESTIMATION

(71) Applicant: Matthew Napier, Cedar City, UT (US)

(72) Inventor: Matthew Napier, Cedar City, UT (US)

(21) Appl. No.: 18/899,921

(22) Filed: Sep. 27, 2024

Related U.S. Application Data
(60) Provisional application No. 63/586,168, filed on Sep. 28, 2023.
(60) Provisional application No. 63/586,177, filed on Sep. 28, 2023.
(60) Provisional application No. 63/586,179, filed on Sep. 28, 2023.

(51) Int. Cl. A61F 13/42

(52) U.S. Cl. CPC A61F 13/42; A61F 2013/424

(57) ABSTRACT

A method for determining diaper saturation. The method for determining diaper saturation includes determining the resistance within the diaper and determining the capacitance within the diaper. The method also includes performing an ADC sample range check and filtering the data. The method further includes performing resistance and capacitance data inferencing.

### CROSS-REFERENCE TO RELATED APPLICATIONS

This application claims the benefit of and priority to U.S. Provisional Patent Application Ser. No. 63/586,177 filed on Sep. 28, 2023, which application is incorporated herein by reference in its entirety.

This application is related to co-pending U.S. patent application Ser. No. ______, filed on Sep. 27, 2024 (Attorney Docket No. 10536-10), and entitled, “THE UTILIZATION OF AN ADAPTABLE LOW POWER SENSOR TO ENABLE SIMULTANEOUS IN-WET EVENT DEVICE 100 HIGH DYNAMIC RANGE MEASUREMENT OF RESISTANCE AND CAPACITANCE TO ESTIMATE AND INTELLIGENTLY TRANSMIT THE SATURATION OF A SENSOR ENHANCED DISPOSABLE DIAPER”, which application is incorporated herein by reference in its entirety (hereinafter “first related application”).

Co-pending application Ser. No. ______ (Attorney Docket No. 10536-10) claims the benefit of and priority to U.S. Provisional Patent Application Ser. No. 63/586,168 filed on Sep. 28, 2023 which application is incorporated herein by reference in its entirety.

This application is related to co-pending U.S. patent application Ser. No. ______, filed on Sep. 27, 2024 (Attorney Docket No. 10536-12), and entitled, “THE UTILIZATION OF MULTIVARIABLE REAL-TIME & CALIBRATION DATA INFERENCING TO IMPROVE THE PREDICTION OF A DISPOSABLE DIAPER'S SATURATION LEVEL”, which application is incorporated herein by reference in its entirety (hereinafter “first related application”).

Co-pending application Ser. No. ______ (Attorney Docket No. 10536-12) claims the benefit of and priority to U.S. Provisional Patent Application Ser. No. 63/586,179 filed on Sep. 28, 2023 which application is incorporated herein by reference in its entirety.

### BACKGROUND OF THE INVENTION

The number of incontinent individuals in the world is rising significantly encompassing the aging population, babies/toddlers, and those who are handicapped/disabled. A cost-effective way to estimate the saturation of a disposable diaper will significantly enhance the quality of life for those people with significant incontinence. Quick changes of a diaper, after an incontinent event, will reduce the risk of skin irritation, UTI's and other diaper related ailments. What is needed is a simple and cost-effective sensor placed directly into the diaper which gives the user or a caregiver information about diaper wetness/saturation. The removable sensor attaches via snaps (or other attachment method) which allows quick and easy diaper changes. Our advanced sensing methods enable this cost-effective solution at a price point lower than competitors.

Sensing the saturation of a diaper with one sensor modality makes it very difficult to repeatedly predict the saturation of a diaper. This multi sensing modality software, when married to a multi sensing front-end sensor, enables multiple sensing modalities to be generated, transmitted and received via IoT message and inferred into a diaper saturation estimate.

### BRIEF SUMMARY OF SOME EXAMPLE EMBODIMENTS

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

One example embodiment includes a method for determining diaper saturation. The method includes determining the resistance within the diaper and determining the capacitance within the diaper. The method also includes performing an ADC sample range check and filtering the data. The method further includes performing resistance and capacitance data inferencing.

Another example embodiment includes a method for determining diaper saturation. The method includes determining the resistance within the diaper. Determining the resistance within the diaper includes setting a sensor to a default state and measuring the voltage of the diaper multiple times to create a voltage data set. Determining the resistance within the diaper also includes pruning the voltage data set and calculating the mean and variance of the pruned diaper plus voltage data set. Determining the resistance within the diaper further includes determining the correct pull-up resistance to be used based on the mean of the voltage data set and calculating the diaper resistance and the parallel resistance of the selected pull-up resistor and the diaper resistance from the current pruned voltage data set. Determining the resistance within the diaper additionally includes disabling all inputs. The method also includes determining the capacitance within the diaper. Determining the capacitance within the diaper includes making a threshold determination of the RC time constant voltage and performing an RC waveform measurement. Determining the capacitance within the diaper also includes determining an RC time constant from the RC waveform measurement and performing a linear interpolation using equation. Determining the capacitance within the diaper further includes repeating the above steps to create a capacitance data set and pruning the capacitance data set. Determining the capacitance within the diaper additionally includes calculating the mean, mode and variance of the pruned diaper plus capacitance data set. The method further includes performing an ADC sample range check and filtering the data. The method additionally includes performing resistance and capacitance data inferencing.

Another example embodiment includes a system for determining a wet event in a diaper. The system includes a front-end sensor configured to be placed within a diaper. The front-end sensor is configured to allow a simultaneous estimation of the resistance within the diaper and the capacitance within the diaper. The system also includes a monitor. The monitor is configured to connect to the front-end sensor and includes a microcontroller unit. The microcontroller is configured to determine the resistance within the diaper and determine the capacitance within the diaper. The microcontroller is also configured to perform an ADC sample range check and filter the data. The microcontroller is further configured to perform resistance and capacitance data inferencing.

These and other objects and features of the present invention will become more fully apparent from the following description and appended claims, or may be learned by the practice of the invention as set forth hereinafter.

### BRIEF DESCRIPTION OF THE DRAWINGS

To further clarify various aspects of some example embodiments of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates an example of a block diagram of a front-end sensor that can be used to measure both resistance and capacitance within a diaper;

FIG. 2 is a flow chart illustrating a method of determining diaper saturation

FIG. 3 is a flow chart illustrating a method for measuring the resistance within a diaper; and

FIG. 4 illustrates a method of determining the capacitance within a diaper.

### DETAILED DESCRIPTION OF SOME EXAMPLE EMBODIMENTS

Reference will now be made to the figures wherein like structures will be provided with like reference designations. It is understood that the figures are diagrammatic and schematic representations of some embodiments of the invention, and are not limiting of the present invention, nor are they necessarily drawn to scale.

The invention is a method that allows measurement of resistance and capacitance using a front-end sensor. A series of measurements are made which allows for estimation of resistance and capacitance within the diaper. Rather than a single measurement, a data set is produced allowing for analysis of diaper environment changes over time.

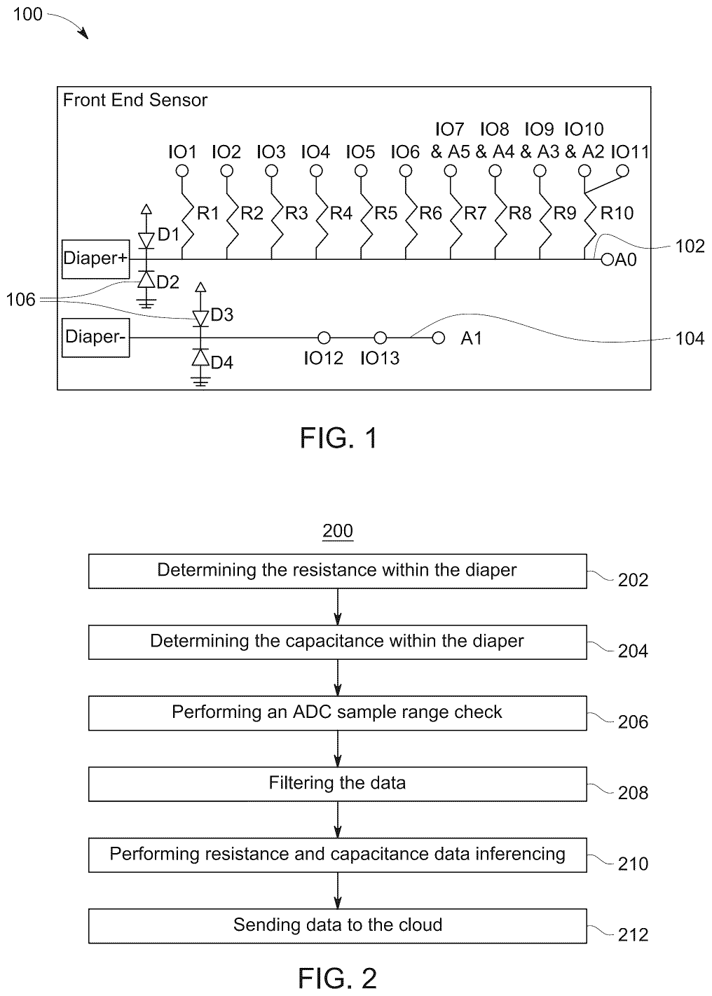

FIG. 1 illustrates an example of a block diagram of a front-end sensor 100 that can be used to measure both resistance and capacitance of the within a diaper. The front-end sensor 100 is placed within the diaper either as an add-on or built into the diaper. In particular, the front-end sensor 100 is placed within the diaper environment. The front-end sensor 100 can be built into the diaper and sold as a single unit or can be sold as a separate device which is placed within a diaper by a user.

FIG. 1 also shows that the front-end sensor 100 can include a first conducting strip 102. The first conducting strip 102 runs the length of the diaper (front to back). The first conducting strip 102 includes a pair of diodes and a series of resistors connected to input/outputs.

FIG. 1 further shows that the front-end sensor 100 can include a second conducting strip 104. The second conducting strip 104 runs the length of the diaper (front to back). The second conducting strip 104 includes a pair of diodes and a series of inputs/outputs.

The combination of the first conducting strip 102 and the second conducting strip 104 allows for the estimation of the resistance within the diaper environment. This allows the wet event device 100 to determine when resistance within the diaper environment has changed. Moisture within the diaper environment, especially sudden changes in moisture, changes the resistance. However, a change in resistance by itself doesn't indicate a wet event because other changes in the environment can cause a change in resistance. For example, if the user is sweating and the sweat runs down the back of the diaper and interacts with the front-end sensor 100, the resistance measurement would be similar to the resistance change due to a wet event. However, this would not require a diaper change. Therefore, changes in resistance as measured by the first conducting strip 102 can indicate a wet event but are not by themselves definitive.

Likewise, the combination of the first conducting strip 102 and the second conducting strip 104 allows for an estimate of the capacitance within the diaper environment. This allows the wet even device 100 to determine when capacitance within the diaper environment has changed. Moisture within the diaper environment, especially sudden changes in moisture, changes the capacitance. However, a change in capacitance by itself doesn't indicate a wet event because other changes in the environment can cause a change in capacitance. For example, in the swat example above, the sweat would create a combination of low resistance and low capacitance whereas a wet event would create low resistance and high capacitance. Further, someone standing in a wet diaper would lead to a different capacitance measurement than that same person sitting in that same diaper. Therefore, changes in capacitance as measured by the second conducting strip 104 can indicate a wet event but are not by themselves definitive.

FIG. 1 additionally shows that the front-end sensor 100 can include electrostatic discharge (ESD) clamp diodes 106 on the first conducting strip 102 and the second conducting strip 104. The ESD clamp diodes 106 protect the first conducting strip 102 and the second conducting strip 104 from the harsh environment of the diaper. In particular, the ESD clamp diodes 106 prevent stray electrical signals (e.g., from the user's body, or from static electricity from rubbing against clothing or the user's body) from creating false signals and damage to the first conducting strip 102 and the second conducting strip 104. In some cases, the Diaper Minus connection is tied directly to sensor ground which removes the need for the ESD clamp diodes 106. Sensor ground is a common reference point for all electronics on the sensor and is not to be confused with earth ground.

The first conducting strip 102 and the second conducting strip 104 can create a multi-component pull up resistance circuit which enables: (1) a high dynamic range diaper resistance measurement estimate, (2) the in-wet event device 100 step response parallel Thevenin equivalent resistance estimation and (3) a high dynamic range capacitance measurement estimate. A high dynamic range pull up resistance circuit enables high dynamic range capacitance measurement estimates in the presence of parallel diaper resistance. High dynamic range measurement of both the resistance and the capacitance allows the inference of the diaper over a wide range of states, from disconnected through fully saturated.

FIG. 2 is a flow chart illustrating a method 200 of determining diaper saturation. In at least one implementation, the method can use a sensor, such as the front-end sensor 100 of FIG. 1. Therefore, the method 200 will be described, exemplarily, with reference to the front-end sensor 100 of FIG. 1. Nevertheless, one of skill in the art can appreciate that the method 200 can be used to produce a determination about diaper saturation using a front-end sensor other than the front-end sensor 100 of FIG. 1.

Collecting simultaneously the steady state (resistance) and dynamic state (capacitance) of a diaper enables measurement of the diaper in two separate sensing modalities. The combination of these two sensing modalities provides additional insight into the diaper state that the individual measurements don't provide. Utilizing both the resistance and capacitance measurement of a diaper the reliability and accuracy of the diaper's saturation level can be significantly enhanced across a diaper's dynamic range.

Combining the real-time measurements of the diaper's distributed resistance and capacitance enables an enhanced capability to estimate the diaper's current saturation level. Variations in urine sodium chloride concentration change both the resistance and capacitance of a diaper in different ways impacting the saturation estimation. Diaper compression (sitting, standing, laying . . . ) impacts capacitance and resistance differently than a wet event impacting the saturation estimation. In addition, sweat and other bodily functions can change the salinity and moisture levels within the diaper. Automatic determination of a diaper's connection status and real-time variations in the capacitance and resistance allows the system to determine whether or not the sensor is connected to a diaper, disconnected or fully saturated. Measurement of just resistance alone or capacitance alone would not allow for differentiation of the above conditions.

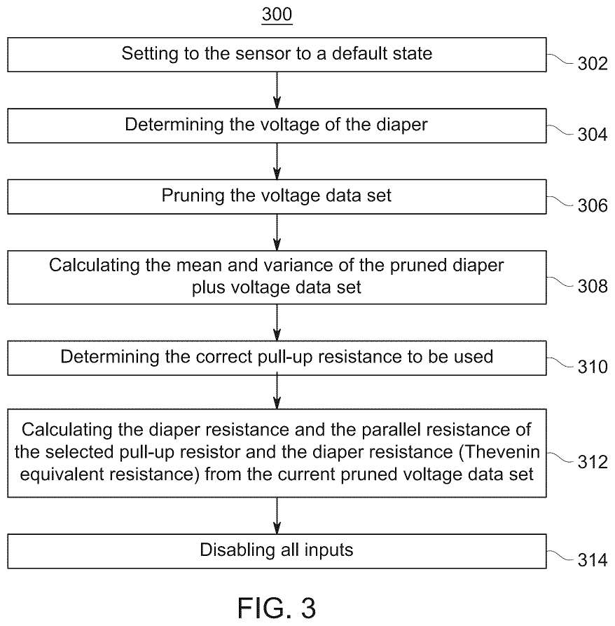

FIG. 2 shows that the method 200 can include determining 202 the resistance within the diaper. Resistance measurements within electrical components is straightforward. When measuring resistance, a small known current, usually in the microampere (μA) range, passes through the component under test. The multimeter then measures the voltage drop across the component. Using Ohm's law, resistance is calculated from the measured voltage and the known current. However, while this would work within a diaper environment it is inadequate. This is because using pull-ups to measure the resistance and using that to create an RC waveform allows for simultaneous estimation of both the resistance and capacitance. Details regarding a possible method of determining 202 the resistance within the diaper are disclosed below and in FIG. 3.

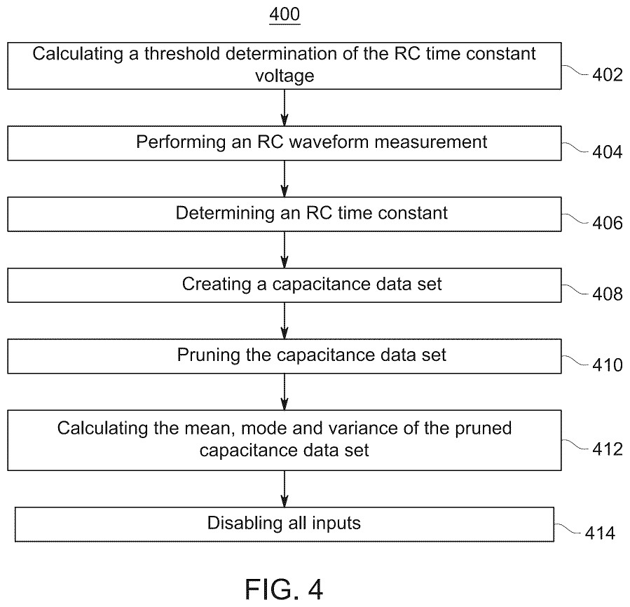

FIG. 2 also shows that the method 200 can include determining 204 the capacitance within the diaper. I.e., once the resistance within the diaper is determined 202, the algorithm then passes the voltage, resistances (i.e., the value of the pull-up resistance being used) and GPIO settings to the capacitance measurement function. Details regarding a possible method of determining 204 the capacitance within the diaper are disclosed below and in FIG. 4.

FIG. 2 also shows that the method 200 can include performing 206 an ADC sample range check. In particular, the number of ADC samples required to estimate the capacitance is checked. If the pull up resistance is too large the RC time constant can become very long, with a shallow slope, which results in a lot of ADC samples which can result in a time constant that is not repeatable. On the opposite side if the pull up resistance is too small, two samples or less, the capacitance measurement can be quite noisy. The ADC sample range check allows immediate introspection of the RC time constant measurement and if it will be repeatable. Inferencing the measurement data real-time is another way to determine if the system is configured correctly to get the best measurement possible. The number of ADC samples up the RC waveform is one of these measurements. Therefore, if the ADC sample range check is performed 206 and the number of samples exceeds 1000 the system is not set up correctly and the method 200 will be started over.

FIG. 2 additionally shows that the method 200 can include filtering 208 the data. I.e., the resistance and capacitance data sets are filtered 208. Both the resistance and capacitance measurements can be noisy due to the noisy diaper environment. Creating filtered versions of these values can help identify how the diaper environment is changing over time. In particular, two filters can be employed, one low pass and one high pass. The low pass filter removes sample to sample noise and provides data that is slower moving and more consistent. The other filter is a high pass filter, it tracks more real time levels of the diaper and is more prone to noise but also does a good job triggering on a diaper's initial wet event. Two infinite impulse response filters are utilized to track how the resistance and capacitance change over time. The electrically dynamic environment of a diaper can result in resistance and capacitance measurements that change significantly from sample to sample. For example, ESD discharge during a capacitance measurement will result in a capacitance measurement that is far removed from the current mean. This sample-to-sample variability is normal in a diaper. The filtering of the measurements enables us to see both short term and long-term trends of the diaper, providing valuable insight. Two-time constants are used. The first-time constant is a fast time constant; the filter tracks the real time data fairly closely using a high pass filter. The second time constant is a slow time constant; the filter tracks the real time data slowly using a low pass filter. The filtered results are compared to the real-time results to determine the current state of the diaper. Filter coefficients are configurable via IoT update.

FIG. 2 moreover shows that the method 200 can include performing 210 resistance and capacitance data inferencing. Inferencing the current and filtered measurements of the resistance and capacitance enables the in-system determination of the diaper state and whether power resources should be utilized to send the current measurements to the cloud. The monitor/sensor will look at both the change of the resistance and capacitance against the previous value and the filtered values and determine if enough change has occurred indicating a new wet event is present and should be messaged to the back-end saturation estimation algorithm. If the change is not great enough the monitor/sensor will not transmit the results to the back end.

Four macro states of the diaper are determined: 1) sensor disconnected from a diaper; 2) sensor connected to the diaper and diaper is dry; 3) sensor connected to diaper and diaper is wet due to sweat; and 4) sensor is connected to a wet diaper. Comparing the above states to previous states enables further insight into the diapers transient state. For example, if the diaper is connected and dry but the previous measurement(s) was disconnected or connected to a wet diaper the sensor determines the diaper was likely changed and triggers a diaper change notification that is sent to the cloud. If the diaper is wet and the change in wetness from the previous measurement(s) and filters exceeds a change threshold the sensor generates a saturation notification and sends a message to the cloud.

FIG. 2 also shows that the method 200 can include sending 212 data to the cloud. The data sent 212 to the cloud includes critical measurements for the back-end processing to do additional analysis on the data. The pruned mean of the resistance and capacitance is sent, along with the filtered 208 data, variances, pull up resistance and ADC sample. Utilizing a third party IoT board support package the messages are transmitted to the cloud via WiFi or Bluetooth connection.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

FIG. 3 is a flow chart illustrating a method 300 for measuring the resistance within a diaper. Measuring the resistance within a diaper can help detection of a wet event. However, the resistance within a diaper can change for a number of reasons. For example, if the user is sweating, then the sweat will be absorbed into the diaper, changing the resistance over time. I.e., a wet event will cause a change in resistance, but not all changes in resistance indicate a wet event.

FIG. 3 shows that the method 300 can include setting 302 the sensor to a default state. The default state creates a known beginning point and allows measurements to begin. The default state selects the pull up resistor w/the highest resistance (R1). To select R1, the pull up GPIO is enabled high (IO1) while all other pull up's GPIOs in the pull up tree are tri-stated (put in a state where they are not driving input or output and thus do not have an impact on the rest of the circuit).

FIG. 3 also shows that the method 300 can include determining 304 the voltage of the diaper. The currently selected pull up resistor will result in a voltage divider circuit between the current pull up resistor and the diaper resistance. The voltage across the diaper is directly dependent on the pull up resistor and the diaper resistance. I.e., the voltage across the diaper is provided by the inputs. If you measured the voltage between the I/O output that is driving the current pull up high and the sensor reference ground you would measure the full voltage range of that I/O. If you measured between the I/O output and the node between the pull up resistance and the diaper plus you would measure a voltage that is a certain portion of the entire voltage range. Then if you measured the voltage between the diaper+node and the diaper-(sensor ground), you would measure the remaining part of the full potential. Summing the former two measurements together will sum to the full voltage applied by the I/O input. How that voltage is distributed across the pull-up and the diaper resistance is directly correlated to the current pull up and the wetness of the diaper. The pull up resistor and diaper resistance are your classic voltage divider circuit. With knowledge of the pull up resistor and the diaper voltage we can calculate the diaper resistance. A statistically significant voltage measurement of the diaper is required due to the dynamic and noisy environment of the diaper. The voltage at the Diaper Plus location (Diaper+) is measured numerous times with a microcontroller unit's (MCU) analog to digital converter (ADC) to create a voltage data set.

FIG. 3 further shows that the method 300 can include pruning 306 the voltage data set. The voltage data set is pruned 306 to remove outliers/noise values improving the variance of the data. To prune 306 the voltage data set, the voltage measurements are sorted from low to high. One way to prune 306 the voltage data set is to remove fixed numbers of minimum and maximum values from the dataset. This ensures that the highest and lowest measurements are removed. I.e., the highest and lowest measurements are assumed to be outliers and rejected. Alternatively, a certain percentage of values can be pruned 306. For example, ten percent of the values can be removed, the highest five percent and the lowest five percent.

FIG. 3 additionally shows that the method 300 can include calculating 308 the mean and variance of the pruned diaper plus voltage data set. The calculation 308 for the mean is well known, and the variance is the square of the standard deviation. Variance is a measure of dispersion, meaning it is a measure of how far a set of numbers is spread out from their mean value. Thus the variance can be used to determine whether a measurement is usable or too noisy to be of use.

FIG. 3 moreover shows that the method 300 can include determining 310 the correct pull-up resistance to be used. To improve both the resistance and capacitance measurements the Diaper+voltage is measured to determine if the correct pull up resistance is selected. The inferred voltage is then compared to a voltage threshold. The voltage threshold is unique for each pull up resistor and can be updated via IoT after device deployment. If the diaper voltage is less than the threshold, then the pull up isn't “strong”/small enough for the current diaper conditions and the algorithm increments to the next smaller pull up resistance and collects voltage measurements per the step above. The process continues through until the inferred diaper voltage is greater than the current pull up resistor threshold. In contrast, if the diaper voltage is greater or equal to the threshold then the pull up currently selected is correct.

FIG. 3 also shows that the method 300 can include calculating 312 the diaper resistance and the parallel resistance of the selected pull-up resistor and the diaper resistance (Thevenin equivalent resistance) from the current pruned voltage data set. The pull up resistance and diaper resistance are measured directly above. Both resistances are the “steady state” resistances of the diaper, also referred to as the DC state of the circuit. In this steady state the pull up resistance and diaper resistance are in series; this is why we can use the voltage divider equation to determine the diaper resistance.

However, measuring the capacitance of the diaper requires a dynamic measurement. An input low to high voltage step wave form is input at and by pull up resistor's I/O. During this dynamic event the diaper circuit looks fundamentally different than when it is in a DC steady state. During this dynamic (temporary state) both the pull up I/O node and the ground node are tied to ground, effectively putting the pull up resistance and diaper resistance in parallel. The resulting parallel resistance value is known as the Thevenin equivalent resistance and is critical when determining the capacitance estimate (as described below).

FIG. 3 further shows that the method 300 can include disabling 314 all inputs. Once the voltage measurements are completed, all GPIO used in the measurement are disabled 314 to optimize power savings. That allows for more measurements before charging is needed and/or for a smaller battery, and therefore a smaller unit needed to make measurements. Further, this gives a known starting point for all measurements.

FIG. 4 illustrates a method 400 of determining the capacitance within a diaper. Capacitance is the capability of a material object or device to store electric charge. It is measured by the charge in response to a difference in electric potential, expressed as the ratio of those quantities. The capacitance of the diaper changes due to the volume of urine in the diaper. However, there are other things that can cause a change in capacitance. I.e., a wet event results in a change of capacitance, but not all changes in capacitance are indicative of a wet event.

FIG. 4 shows that the method 400 can include calculated 402 a threshold determination of the RC time constant voltage. Critical to the capacitance measurement is the RC time constant voltage threshold. The threshold determination is calculated 402 from the combination of the real-time in-system voltages and resistance measurements and derived Thevenin resistance.

FIG. 4 further shows that the method 400 can include performing 404 an RC waveform measurement. Performing 404 an RC waveform measurement is critical to estimating the diaper's current capacitance as it allows the diaper's RC time constant to be determined. To perform the RC time constant, the currently selected pull-up resistor general GPIO is driven low to high, putting an impulse into the system. The current time and voltage at the Diaper Plus connection is measured. The measurement is compared to a threshold and if the voltage has not exceeded the threshold another measurement is taken until the threshold has been met. The previous (less than threshold) and current (greater than the threshold) voltage and time measurement are stored. The previous (less than threshold) voltage is saved as variable x2 and time measurement is saved as variable y2 and the current (greater than the threshold) voltage is saved as variable x1 and time measurement is saved as variable y1.

FIG. 4 additionally shows that the method 400 can include determining 406 an RC time constant. From those two voltage and time measurements (i.e., x1, x2, y1 and y2), a time constant is determined. Interpolation of the current and previous measurement around the threshold is calculated to increase the time constant accuracy. Linear interpolation is calculated using equation 1.

$$y = y 1 + ( ( x - x 1 ) / ( x 2 - x 1 ) ) * ( y 2 - y 1 ) \qquad Equation l$$

y is the time the Diaper Plus voltage passed the voltage threshold (x in the equation above). The equation is then solved for y resulting in a time constant. More advanced interpolation methods could also be employed to improve interpolation accuracy.

The RC time constant, denoted y in equation 1, the time constant (in seconds) of a resistor-capacitor circuit (RC circuit), is equal to the product of the circuit resistance (in ohms) and the circuit capacitance (in farads). One of skill in the art will appreciate that if the resistance can be measured (or estimated) and the RC time constant can be measured (or estimated), then the capacitance can be calculated. From the time constant, the capacitance can be estimated. Measuring the time constant results in time_constant/RC=1 and DiaperCapacitanceEst=RCTimeConst/TheveninResistance).

FIG. 4 moreover shows that the method 400 can include creating 408 a capacitance data set. As with the voltage, the steps 402 through 406 are repeated to create 408 the data set. One measurement could be used, but creating 408 a capacitance data set creates a more statistically significant measurement. In particular, one of skill in the art will appreciate that a statistically significant capacitance measurement of the diaper is required due to the dynamic and noisy environment of the diaper.

FIG. 4 moreover shows that the method 400 can include pruning 410 the capacitance data set. The capacitance data set is pruned 410 to remove outliers/noise values improving the variance of the data. To prune 410 the capacitance data set, the capacitance measurements are sorted from low to high. One way to prune 410 the capacitance data set is to remove fixed numbers of minimum and maximum values from the dataset. This ensures that the highest and lowest measurements are removed. I.e., the highest and lowest measurements are assumed to be outliers and rejected. Alternatively, a certain percentage of values can be pruned 410. For example, ten percent of the values can be removed, the highest five percent and the lowest five percent.

FIG. 4 also shows that the method 400 can include calculating 412 the mean, mode and variance of the pruned capacitance data set. The calculation 412 for the mean and mode are well known, and the variance is the square of the standard deviation. Variance is a measure of dispersion, meaning it is a measure of how far a set of numbers is spread out from their mean value.

FIG. 4 further shows that the method 400 can include disabling 414 all inputs. Once the voltage measurements are completed, all GPIO used in the measurement are disabled 414 to optimize power savings. That allows for more measurements before charging is needed and/or for a smaller battery, and therefore a smaller unit needed to make measurements. Further, this gives a known starting point for all measurements.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method for determining diaper saturation, the method comprising:
determining the resistance within the diaper;
determining the capacitance within the diaper;
performing an ADC sample range check;
filtering the data; and
performing resistance and capacitance data inferencing.

2. The method of claim 1, wherein resistance and capacitance data inferencing is used to determine the macro state of a diaper.

3. The method of claim 2, wherein the macro state of the diaper includes sensor disconnected from a diaper.

4. The method of claim 2, wherein the macro state of the diaper includes sensor connected to the diaper and diaper is dry.

5. The method of claim 2, wherein the macro state of the diaper includes sensor connected to diaper and diaper is wet due to sweat.

6. The method of claim 2, wherein the macro state of the diaper includes sensor is connected to a wet diaper.

7. The method of claim 2, wherein the macro state of the diaper is sent to a user.

8. A method for determining diaper saturation, the method comprising:
determining the resistance within the diaper, wherein determining the resistance within the diaper includes:
setting a sensor to a default state;
measuring the voltage of the diaper multiple times to create a voltage data set;
pruning the voltage data set;
calculating the mean and variance of the pruned diaper plus voltage data set;
determining the correct pull-up resistance to be used based on the mean of the voltage data set;
calculating the diaper resistance and the parallel resistance of the selected pull-up resistor and the diaper resistance from the current pruned voltage data set; and
disabling all inputs;
determining the capacitance within the diaper, wherein determining the capacitance within the diaper includes:
making a threshold determination of the RC time constant voltage;
performing an RC waveform measurement;
determining an RC time constant from the RC waveform measurement;
repeating the above steps to create a capacitance data set;
pruning the capacitance data set; and
calculating the mean, mode and variance of the pruned diaper plus capacitance data set;
performing an ADC sample range check;
filtering the data; and
performing resistance and capacitance data inferencing.

9. The method of claim 8 wherein pruning the voltage data set includes sorting the voltage data set from low to high.

10. The method of claim 9 wherein pruning the voltage data set includes removing fixed numbers of the lowest voltage measurements and fixed numbers of the highest voltage measurements.

11. The method of claim 8 wherein pruning the capacitance data set includes sorting the capacitance data set from low to high.

12. The method of claim 11 wherein pruning the capacitance data set includes removing fixed numbers of the lowest capacitance measurements and fixed numbers of the highest capacitance measurements.

13. A system for determining a wet event in a diaper, the system comprising:
a front-end sensor configured to be placed within a diaper, wherein the front-end sensor is configured to allow a simultaneous estimation of:
the resistance within the diaper; and
the capacitance within the diaper; and
a monitor, wherein the monitor:
is configured to connect to the front-end sensor; and
includes a microcontroller unit, wherein the microcontroller is configured to:
determine the resistance within the diaper;
determine the capacitance within the diaper;
perform an ADC sample range check;
filter the data; and
perform resistance and capacitance data inferencing.

14. The method of claim 13 wherein data inferencing includes determining whether moisture level of the diaper has changed enough to send a message to a user.

15. The method of claim 13 further comprising a communications block.

16. The method of claim 15 wherein the communications block includes:
Bluetooth connectivity; and
Wi-Fi connectivity.

17. The method of claim 16, wherein the communications block is configured to send the inferenced data to a user.

18. The method of claim 13, wherein performing an RC waveform measurement includes linear interpolation using equation 1.
